(19) United States
(12) Patent Application Publication
(10) Pub. No.: US 2023/0245745 A1
(43) Pub. Date: Aug. 3, 2023

(54) ALARM FOR IMPLANTABLE DEVICE STOPPED TOO LONG FOR A PROGRAMMING UPDATE

(71) Applicant: Medtronic, Inc., Minneapolis, MN (US)

(72) Inventors: Earle T. Roberts, Plymouth, MN (US); Bruce L. Brasaemle, Duluth, MN (US); Touby A. Drew, Eden Prairie, MN (US)

(21) Appl. No.: 18/099,638

(22) Filed: Jan. 20, 2023

Related U.S. Application Data
(60) Provisional application No. 63/305,886, filed on Feb. 2, 2022.

(51) Int. Cl. G16H 20/17

(52) U.S. Cl. CPC G16H 20/17; A61M 2205/18

(57) ABSTRACT

An implantable medical device is configured to alert a user of a failed or delayed automatic restart following a programming update. In some examples, the device is configured to wirelessly receive a programming update that includes a command instructing the implantable medical device to cease a therapy-delivery regimen while installing the programming update, and a timeout module configured to initiate a countdown timer for a predetermined duration of time, whereupon failure of the regimen to automatically restart upon expiration of the predetermined duration of time triggers an alert to notify a user that the implantable medical device has failed to restart.

### PRIORITY CLAIM

The present application claims the benefit of U.S. Provisional Patent Application No. 63/305,886, filed Feb. 2, 2022, and titled “ALARM FOR IMPLANTABLE DEVICE STOPPED TOO LONG DURING A PROGRAMMING UPDATE,” the entire contents of which are incorporated herein by reference

### TECHNICAL FIELD

The present technology is generally related to implantable medical devices.

### BACKGROUND

Implantable medical devices, such as implantable medical pumps and ports, are useful in managing the delivery and dispensation of prescribed therapeutic agents, nutrients, drugs, infusates such as antibiotics, blood-clotting agents, analgesics, and other fluid or fluid-like substances (collectively “infusate” or “infusates”) to patients in volume-controlled and time-controlled doses as well as through boluses. Such implantable pumps and ports are particularly useful for treating diseases and disorders that require regular or chronic (i.e., long-term) pharmacological intervention, including tremor, spasticity, multiple sclerosis, Alzheimer's disease, Parkinson's disease, amyotrophic lateral sclerosis (ALS), Huntington's disease, cancer, epilepsy, chronic pain, urinary or fecal incontinence, sexual dysfunction, obesity, and gastroparesis, to name just a few. Depending upon their specific designs and intended uses, implantable pumps and ports are well-adapted to administer infusates to specific areas within the vasculature and central nervous system, including the subarachnoid, epidural, intrathecal, and intracranial spaces, or to provide access to one or more of those spaces for aspiration of unwanted material.

Providing access to the cerebrospinal fluid for the administration of infusates or aspiration of fluid has a number of important advantages over other forms of infusate administration. For example, oral administration is often not workable because the systematic dose of the substance needed to achieve the therapeutic dose at the target site may be too large for the patient to tolerate without adverse side effects. Also, some substances simply cannot be adequately absorbed in the gut for a therapeutic dose to reach the target site. Moreover, substances that are not lipid-soluble may not cross the blood-brain barrier adequately if needed in the brain. In addition, infusion of substances from outside the body requires a transcutaneous catheter or access with a hypodermic needle, which carries other risks such as infection or catheter dislodgement. Further, implantable pumps avoid the problem of patient noncompliance, namely the patient failing to take the prescribed drug or therapy as instructed.

Implantable medical infusion pumps are typically implanted within the body of a patient (e.g., a subcutaneous region in the lower abdomen), and are configured to deliver a fluid medicament through a catheter. The catheter is generally configured as a flexible tube defining a lumen running the length of the catheter to a selected delivery site in the body, such as the subarachnoid, epidural, intrathecal, and intracranial spaces. Such implantable medical pumps typically include an expandable fluid reservoir, which is accessible for refill, etc., through an access port. Medicament flows from the reservoir via the lumen in the catheter according to programmed parameters.

In some cases, the implantable medical pump can be configured to receive the programmed parameters and other updates from an external programming device, sometimes referred to as a “controller” or a “programmer,” which can communicate with the implantable medical pump through well-known techniques such as wireless telemetry. A clinician or patient can use the external programmer to change the therapy-delivery regimen, for example by defining a treatment protocol. Typically, a clinician or patient interfaces with the external programmer to set various parameters associated with the implantable medical pump, and then transmits or uploads those parameters to the implantable medical pump.

When a treatment protocol or software of the programmable infusion pump is updated, it is often safe or necessary to stop the pump to avoid any transitory effects from affecting the programming update and to ensure that the programming update is automatically applied. However, stopping the pump while a programming update is in-progress creates a potential underdose-hazard situation where the pump could unknowingly be left in an “off” state. Such an interruption can be caused, for example, by the programming head being physically moved out of position, an interruption of the clinician by a colleague, the clinician changing their mind about the intended programming settings, or a “noisy” environment that makes telemetry communication difficult. The present disclosure addresses these concerns.

### SUMMARY OF THE DISCLOSURE

The techniques of this disclosure generally relate to implantable medical devices and systems including a feature wherein, if fluid delivery is paused or stopped for an above-threshold duration for a programming update, the implantable medical device can alert or otherwise notify a clinician or patient of an excessive delay in fluid delivery. Various visual, audible, and tactile alerts and notifications are contemplated. Upon interrogating the implantable device with an external programmer, the implantable device can report that fluid delivery was stopped for a programming update, and remains stopped. By providing this feedback shortly after the completion of a programming update, a high-severity hazard can be avoided.

Some examples of the present disclosure include medical systems configured to provide an alert to notify a user that an implantable medical device has failed to restart following a programming update, the medical system including an implantable medical device configured to deliver a therapy according to a therapy-delivery regimen, and an external device in wireless communication with the implantable medical device, the external device configured to communicate a programming update to the implantable medical device, wherein the programming update comprises a command instructing the implantable medical device to cease delivery of the therapy while installing the update, and a timeout module configured to initiate a countdown timer for a specified period of time, whereupon failure of the therapy-delivery regimen to restart by an end of the specified period of time triggers an alert to notify a user that the implantable medical device has failed to restart.

In some examples, upon a successful loading of the programming update by the implantable medical device, the therapy-delivery regimen is automatically restarted. In some such examples, upon restarting of the therapy-delivery regimen, the countdown timer is terminated. In some examples, the implantable medical device includes an infusion pump. In some examples, the programming update is configured to provide an update to software loaded on the implantable medical device, or to modify the therapy-delivery regimen (e.g., modify a frequency, dosage amount, etc.). In some examples, the implantable medical device is configured to emit an alarm in the form of an auditory, tactile or visual signal. In some examples, the implantable medical device is configured to wirelessly transmit an alarm in the form of a notification to the external device. In some examples, the specified period of time is 5 minutes, 10 minutes, 15 minutes, 20 minutes, 25 minutes, or 30 minutes.

Some examples of the present disclosure include medical devices configured to provide an alert to notify a user of a failed restart from a programming update, including an implantable medical device configured to wirelessly receive a programming update, wherein the programming update includes a command instructing the implantable medical device to cease delivery of a therapy while installing the programming update, and a timeout module configured to initiate a countdown timer for a specified period of time, whereupon failure of the therapy-delivery regimen to automatically restart by an end of the specified period of time triggers an alert to notify a user that the implantable medical device has failed to restart.

Some examples of the present disclosure provide a method of notifying a user that an implantable medical device has failed to restart following a programming update, including wirelessly transmitting a programming update to an implantable medical device, wherein the programming update comprises a command instructing the implantable medical device to cease delivery of the therapy while installing the programming update, and a timeout module configured to initiate a countdown timer for a specified period of time, whereupon failure of the therapy-delivery regimen to restart by an end of the specified period of time triggers an alert to notify a user that the implantable medical device has failed to restart.

The details of one or more aspects of the disclosure are set forth in the accompanying drawings and the description below. Other features, objects, and advantages of the techniques described in this disclosure will be apparent from the description and the drawings, and from the claims.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a schematic view of an example medical system including an implantable medical device.

FIGS. 2A and 2B are cross-sectional views of an example of the implantable medical device of FIG. 1.

FIG. 3 is a conceptual block diagram of examples of the implantable medical device and external programmer device of the medical system of FIG. 1.

FIG. 4 is a schematic diagram of an example of the medical system of FIG. 1.

FIG. 5 is a flow diagram depicting an example technique for alerting a user of a failed or delayed medicament-delivery restart following a programming update to a medical device.

While examples of this disclosure are amenable to various modifications and alternative forms, specifics thereof shown by way of example in the drawings will be described in detail. It should be understood, however, that the intention is not to limit the disclosure to the particular examples described.

### DETAILED DESCRIPTION

The present disclosure describes various examples of implantable medical devices, systems, and techniques for alerting or otherwise notifying a clinician or patient that delivery of a therapeutic treatment has been stopped or paused beyond a predetermined duration of time, e.g., a length of time that would typically be expected for a given programming update. Although specific examples of implantable medical pumps are provided, it is to be appreciated that the concepts disclosed herein are extendable to other types of implantable medical devices. It is also to be appreciated that the term “clinician” refers to any individual that can prescribe and/or program a therapy-delivery regimen with any of the example examples described herein or combinations thereof. Similarly, the terms “patient” or “subject,” as used herein, are to be understood to refer to an individual or object in which the infusate delivery is to occur, whether human, animal, or inanimate. Various descriptions are made herein, for the sake of convenience, with respect to the procedures being performed by a clinician on a patient or subject (the involved parties collectively referred to as a “user” or “users”), while the disclosure is not necessarily limited in this respect.

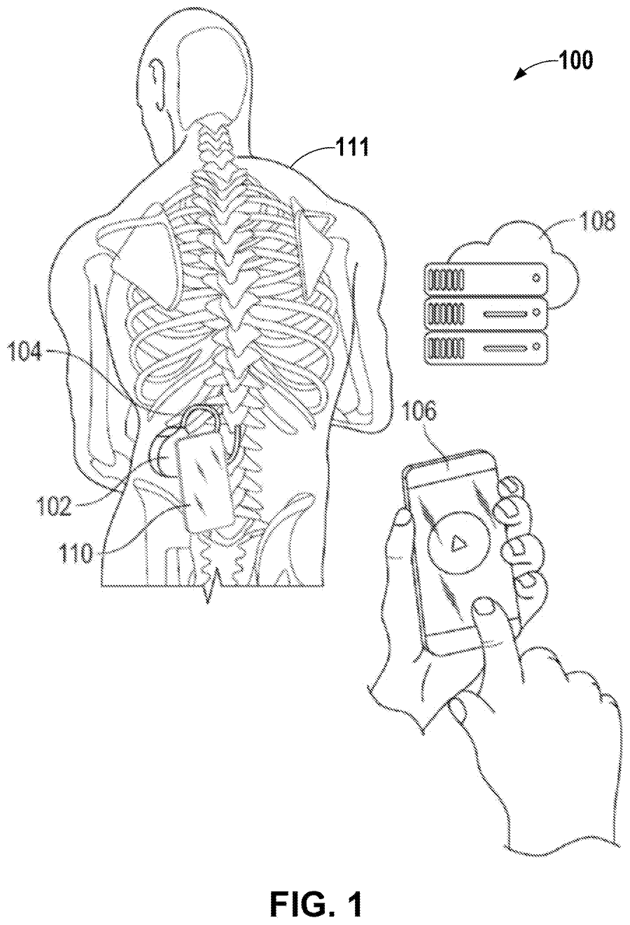

FIG. 1 depicts a medical system 100 including an implantable medical device 102 configured to alert or otherwise notify a clinician or patient that implantable medical device 102 has been stopped or paused beyond an expected length of time for a programming update. In some examples, implantable medical device 102 can be an implantable pump or port configured to dispense infusate over an extended period of time. Other contemplated implantable medical devices include a cardiac pacemaker, cardiac defibrillator, cardiac resynchronization device, cardiac-monitoring device, cardiac-pressure-monitoring device, spinal-stimulation device, neural-stimulation device, gastric-stimulation device, insulin pump, or another drug-delivery device. As depicted in FIG. 1, a clinician can implant device 102 within the body of a patient 111, for example, in an interior torso cavity, in proximity to the patient's ribs, or cranially, for the targeted delivery of an infusate into the patient (e.g., within an intrathecal space, intracranial space, pulmonary artery, etc.). In some examples, implantable device 102 can be placed subcutaneously, and can be held in position by sutures or other retaining features. In some examples, implantable medical device 102 includes an implantable catheter 104 configured to deliver infusate to a particular location in the patient's body.

In some examples, medical system 100 includes an external programmer 106 and optional remote server 108, either or both being configured to communicate with implantable device 102. In some examples, programmer 106 can be a mobile computing device, such as a cellular telephone, tablet, dedicated implantable-device programmer, or the like. Further, in some examples, medical system 100 includes one or more telemetry heads 110 configured to facilitate communication between implantable device 102, external programmer 106, and optional server 108. In other examples, the external programmer 106 and optional server 108 can communicate directly with implantable device 102.

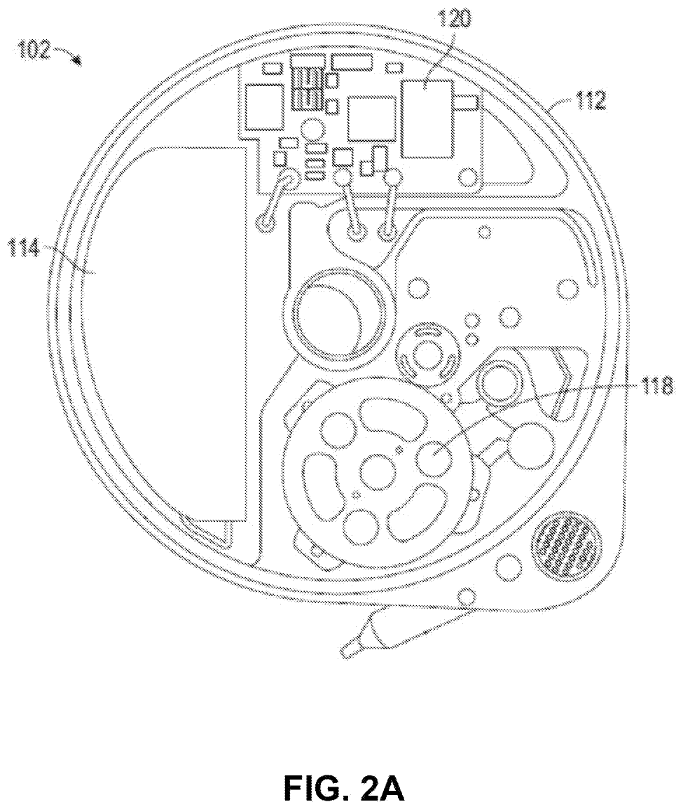

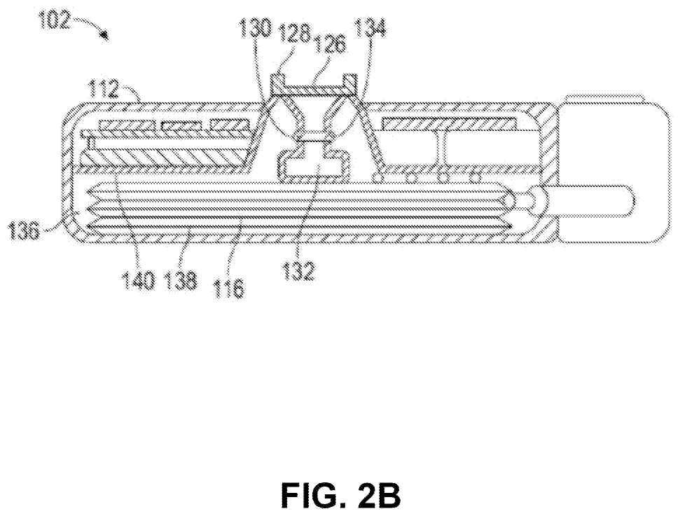

FIGS. 2A and 2B are cross-sectional views of an example of implantable medical device 102 of FIG. 1. Implantable device 102 is configured to alert a user of stopped or paused infusate delivery beyond an expected length of time for a programming update. Implantable device 102 can generally include a housing 112, power source 114, fluid reservoir 116, pump 118, and computing device 120. Housing 112 can be constructed of a material that is biocompatible and hermetically sealed, such as titanium, tantalum, stainless steel, plastic, ceramic, or the like.

Fluid reservoir 116 can be retained within the housing 112 and can be configured to contain infusate. In some examples, a user can access infusate within the reservoir 116 via an access port 126. Accordingly, a user can use access port 126 to refill, aspirate, or exchange fluid within reservoir 116. In some examples, access port 126 includes one or more positional markers 128, for example, tactile protrusions, visible lights (e.g., LEDs) configured to illuminate through tissue of patient 111, acoustic devices (e.g., speakers) to configured to indicate the location of access port 126, or wireless location or orientation sensors configured to aid in positioning of a refilling device relative to implantable device 102.

In some examples, access port 126 includes a septum 130 configured to seal a port chamber 132 relative to an exterior of housing 112. Septum 130 can be constructed of a silicone rubber or other material having desirable self-sealing and longevity characteristics. Port chamber 132 can be in fluid communication with reservoir 116. In some examples, access port 126 includes a needle detector 134 in the form of, for example, a mechanical switch, a resonant circuit, an ultrasonic transducer, a voltmeter, an ammeter, an ohmmeter, a pressure sensor, a flow sensor, a capacitive probe, an acoustic sensor, or an optical sensor configured to detect and indicate the presence of an injection needle of a refilling apparatus.

Fluid reservoir 116 can include a flexible diaphragm 138. Flexible diaphragm 138, alternatively referred to as a “bellows,” can be substantially cylindrical in shape and can include one or more convolutions enabling flexible diaphragm 138 to expand and contract between an extended or “full” position and a contracted or “empty” position. In some examples, flexible diaphragm 138 divides fluid reservoir 116 into an infusate chamber containing liquid infusate (e.g., within the flexible diaphragm 138), and a vapor chamber 136 (e.g., surrounding the flexible diaphragm 138). As the infusate chamber fills with infusate, flexible diaphragm 138 extends downward (from the perspective illustrated in FIG. 2B) toward a bottom portion of housing 112 until it has reached a maximum volume or other desired degree of fullness. Alternatively, as the infusate chamber is aspirated, flexible diaphragm 138 contracts upward toward a top portion of housing 112 until the infusate chamber reaches a minimum volume. In some examples, flexible diaphragm 138 includes a compression spring which tends to naturally bias flexible diaphragm 138 toward an expanded or partially expanded configuration.

Housing 112 can retain pump 118, which can be in fluid communication with fluid reservoir 116 and in electrical communication with computing device 120. Pump 118 can be any pump sufficient for pulling infusate from reservoir 116 for downstream delivery, such as a peristaltic pump, a piston pump, a pump powered by a stepper motor or a rotary motor, a pump powered by an AC motor, a pump powered by a DC motor, an electrostatic diaphragm, a piezoelectric motor, a solenoid, a shape-memory alloy, or the like.

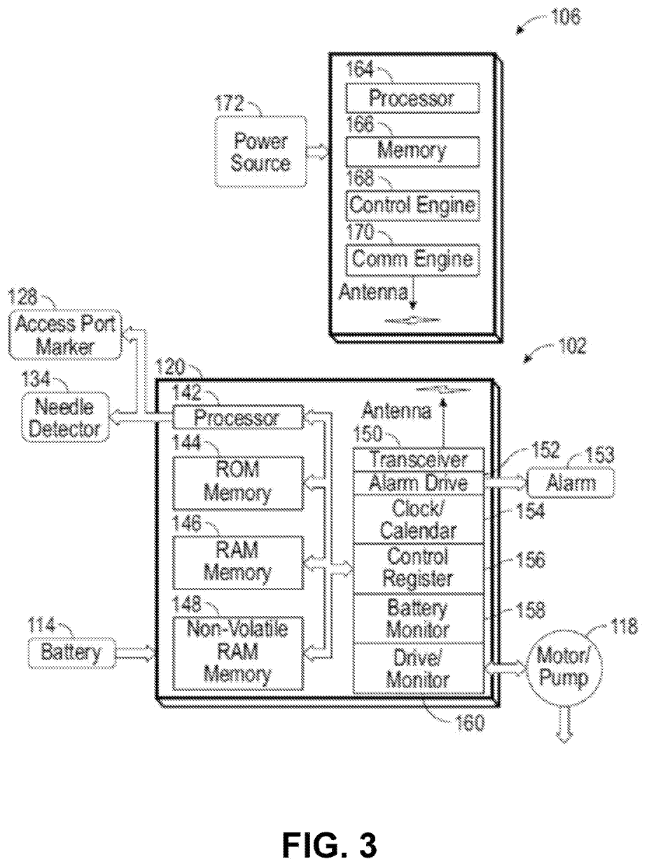

FIG. 3 is a conceptual block diagram of examples of implantable device 102 and external programmer 106 of FIG. 1, configured to alert a user of a stopped or paused infusate delivery for a duration that is beyond an expected length of time for a programming update. Implantable device 102 can include a computing device 120, which can be retained within housing 112 (as depicted in FIG. 2A) and can be in electrical communication with pump 118 and power source 114. In the specific example of FIG. 3, power source 114 is depicted as a battery, such as a rechargeable lithium-ion battery, a nickel-cadmium battery, or the like. Power source 114, which can be monitored via the battery monitor 158, can be retained within housing 112 to power pump 118 and computing device 120. A drive/monitor element 160 can control pump 118.

Computing device 120 can include a processor 142, memory 144, 146, and 148, and transceiver circuitry 150. In some examples, processor 142 can be a microprocessor, logic circuit, Application-Specific Integrated Circuit (ASIC) state machine, gate array, controller, or the like. In general, computing device 120 is configured to control delivery of infusate according to programmed parameters or a specified treatment protocol. The programmed parameters or specified treatment protocol can be stored in memory 144, 146, and 148 for specific implementation by a control register 156. A clock/calendar element 154 can maintain system timing for computing device 120. In some examples, an alarm drive 152 can be configured to activate one or more notification, alert, or alarm features, such as a visual (e.g., illumination-based), auditory, or tactile (e.g., vibration-based) alarm 153.

In some examples, processor 142 can be configured to selectively activate needle detector 134 and access-port marker(s) 128, prior to a physical attempt to insert a needle of the refill device into access port 126 of implantable device 102. Processor 142 can be configured to receive input from drive/monitor element 160, which can aid in monitoring for a pressure decay of infusate within tubing generally associated with catheter 104 downstream of pump 118, e.g., to help detect and diagnose catheter occlusion, dislodgment, kink, or other undesirable but foreseeable conditions within system 100.

For example, the downstream pressure and pressure decay can be inferred from a sensed electromotive force (EMF) voltage of pump 118 by noting movement in the rotor of the pump after power has been removed. In operation of pump 118, drive currents to a stator are selectively applied and removed by drive/monitor element 160. Through computing device 120, a resulting EMF voltage can be sensed in each of a series of stator coils after the drive currents are removed, from which a position of the rotor can be determined.

In particular, the rotor naturally comes to rest at an equilibrium position determined by the magnetic forces between the stator and rotor, as well as external forces (e.g., pressurized fluid within catheter 104). In a normal, un-occluded state, the EMF voltage will fluctuate as the rotor rocks back and forth slightly to settle into an equilibrium position. By contrast, when pump 118 is occluded (and the downstream pressure is elevated), oscillations in the EMF voltage will be diminished, as movement of the rotor is significantly dampened by pressurized medicament trapped downstream. Accordingly, dampened oscillations in sensed EMF voltage can be indicative of slow pressure decay (e.g., occlusion, kinked tubing, other system issues, etc.).

Transceiver circuitry 150 is configured to receive information from, and transmit information to, external programmer 106, server 108, or telemetry head 110. Implantable device 102 can be configured to receive programmed parameters and other updates from external programmer 106, which can communicate with implantable device 102 through well-known techniques such as wireless telemetry, mixed band, Bluetooth, or one or more proprietary communication schemes (e.g., Tel-A, Tel-H, Tel-M, Tel-C, etc.). In some examples, external programmer 106 and cloud-based server 108 are configured to receive, store, and transmit information, such as program parameters, treatment protocols, drug libraries, patient information, and data recorded by implantable device 102.

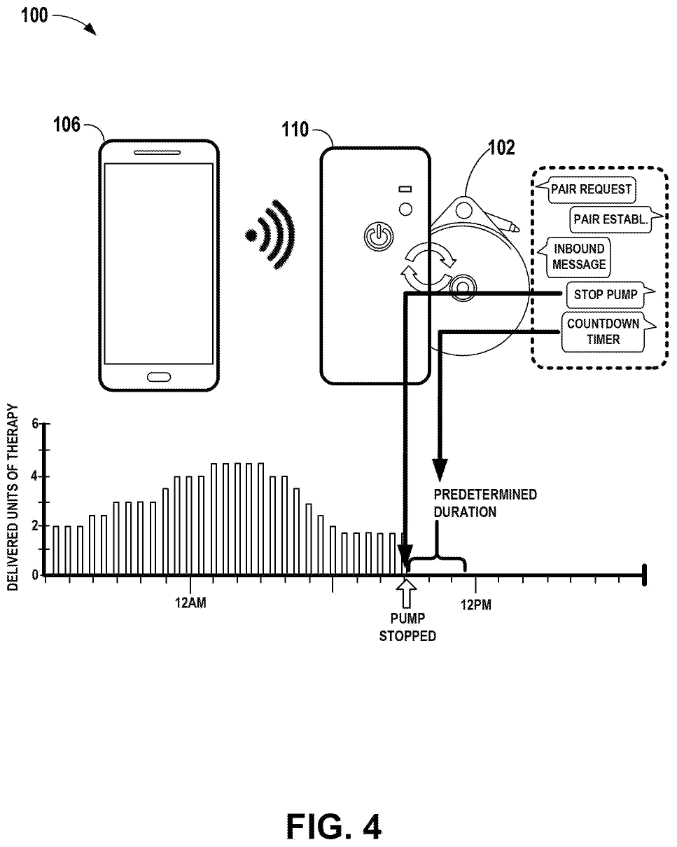

In some examples, external programmer 106 is configured for exclusive communication with one or more of implantable device(s) 102. In other examples, external programmer 106 can be any computing platform, such as a mobile phone, tablet, or personal computer. In some examples, programmer 106 and cloud-based server 108 can communicate directly with implantable device 102. In other examples, implantable device 102 can communicate with programmer 106 and cloud-based server 108, either directly or via telemetry head 110 (FIGS. 1 and 4).

With continued reference to FIG. 3, in some examples, external programmer 106 includes various sub-modules or engines, each of which is constructed, programmed, configured, or otherwise adapted to autonomously carry out a particular function or set of functions. The term “engine” as used herein is defined as a real-world device, component, or arrangement of components implemented using hardware, such as by an application specific integrated circuit (ASIC) or a field-programmable gate array (FPGA), or as a combination of hardware and software, such as by a microprocessor system and a set of associated program instructions that adapt the engine to implement the particular functionality, which (while being executed) transform the microprocessor system into a special-purpose device. An engine can also be implemented as a combination of the two, with certain functions facilitated by designated hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-to-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engine, each of which can be regarded as an “engine” in its own right. Moreover, in examples described herein, each of the various engines corresponds to a defined autonomous functionality, however, it should be understood that, in other contemplated examples, each functionality can be distributed across more than one engine. Likewise, in other contemplated examples, multiple defined functionalities may be implemented by a single engine that performs those multiple functions, possibly alongside other functions, or distributed differently among a set of engines than specifically illustrated and described herein.

As shown in FIG. 3, external programmer 106 can include a processor 164, memory 166, a control engine 168, a communications engine 170, and a power source 172. Processor 164 can include any suitable fixed-function circuitry or programmable processing circuitry, such as a microprocessor, a controller, a DSP, an ASIC, an FPGA, or equivalent discrete or analog logic circuitry. In some examples, processor 164 can include multiple components, such as any combination of one or more microprocessors, one or more controllers, one or more DSPs, one or more ASICs, or one or more FPGAs, as well as other discrete or integrated logic circuitry. The functions attributed to processor 164 herein may be implemented as software, firmware, hardware, or any combination thereof.

Memory 166 can include computer-readable instructions that, when executed by processor 164, cause computing device 120 of medical device 102 to perform various functions. Memory 166 can include volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Control engine 168 can include instructions to control the components of programmer 106 and instructions to selectively control implantable medical device 102.

Communications engine 170 can include any suitable hardware, firmware, software, or any combination thereof, for communicating with other components of medical device 102 and/or external devices. Under the control of processor 164, communications engine 170 can receive downlink telemetry from, as well as send uplink telemetry to, one or more external devices (e.g., implantable medical device 102, etc.) using an internal or external antenna. In addition, communications engine 170 can facilitate communication with a networked computing device and/or a computer network, such as remote server 108. For example, communications engine 170 can receive updates to instructions for control engine 168 from one or more external devices. In another example, communications engine 170 can transmit data regarding the state of system 100 to one or more one or more external devices.

Power source 172 is configured to deliver operating power to the components of external programmer 106. Power source 172 can include a battery and a power-generation circuit to produce the operating power. In some examples, the battery is rechargeable to allow extended operation. Power source 172 can include any of a plurality of different battery types. In some examples, programmer 106 additionally includes an external power-supply port.

FIG. 4 is a schematic diagram of a non-limiting example of medical system 100 of FIG. 1, configured to provide a user alert or notification that infusate delivery has been stopped or paused beyond an expected length of time for a programming update. Communication between implantable device 102 and programmer 106, server 108, or telemetry head 110 (e.g., via a nonproprietary telecommunication technology/protocol) can be facilitated through trusted pairing. For instance, communications to and from implantable device 102 can be restricted to the type of communications authorized between implantable device 102 and external device(s) 106, 108, 110 based on association of external device(s) 106, 108, 110 with a potentially “trusted” device. For example, transceiver 150 can reject all transmissions from external devices 106, 108, 110, except transmissions that include validation information to establish trusted pairing between implantable device 102 and the one or more external devices 106, 108, 110.

In some examples, communication devices such as implantable device 102 and external devices 106, 108, 110 have to agree on many physical-layer and link-layer aspects of the data to be exchanged before a successful connection can be established. Rules defining how to establish a connection and perform telemetry communications are commonly referred to in wireless-communication technology as “protocols.” For instance, in order to establish a secure telemetry connection, many communication protocols require a procedure called “pairing” wherein the respective devices form a trusted relationship prior to being able to communicate certain information with one another. Communication protocols cover authentication, error detection and correction, and signaling. Communication protocols are implemented in hardware and software. As used herein, the term “non-proprietary telemetry communication technology/protocol” refers to any standardized telemetry communication technology/protocol that can be commercially used by more than one entity, either in an open-source context or a licensed context. Many non-proprietary telemetry communication technologies/protocols have publicly available specifications.

In some examples, implantable device 102 can generate a prompt that requests the user provide input (e.g., a username or password, a pass-code, a secret key, etc.) that verifies the user's identity and/or verifies that the user is authorized to pair with implantable device 102. Implantable device 102 can further have access to information stored in a memory of external devices 106, 108, 110 that associates the required user input with an authorization to pair with implantable device 102.

After implantable device 102 and external devices 106, 108, 110 have paired and established a secure telemetry connection, implantable device 102 can use a non-proprietary telemetry protocol to exchange various types of information with external devices 106, 108, 110. For example, using a non-proprietary telemetry protocol, implantable device 102 can transmit information to external devices 106, 108, 110. In another example, using a non-proprietary telemetry protocol, external devices 106, 108, 110 can send information or signals to implantable device 102 to program implantable device 102 or to configure (or re-configure) implantable device 102.

Once trusted pairing has been established, communication between implantable device 102 and external devices 106, 108, 110 can occur in the form of messages (sometimes referred to as “communication signals” or “packets”) that are passed back-and-forth between the devices. With regard to communications, the term “inbound” generally refers to activities associated with telemetry reception by medical device 102, whether implanted or not, or to activities associated with telemetry transmission by external devices 106, 108, 110; the term “outbound” generally refers to activities associated with the telemetry transmission by medical device 102, whether implanted or not, or to activities associated with telemetry reception by external devices 106, 108, 110.

In some examples, these messages can have a multi-part format or protocol, including: (1) preamble, (2) frame sync, (3) telemetry identifier, and (4) data; although other example multi-part formats are also contemplated. For instance, the multi-part format may include (1) a preamble, (2) a telemetry identifier, and (3) data. In other examples, the preamble and frame sync may be combined into a single entity, or the telemetry identifier and the frame sync may be combined into a single entity, or even all of the preamble, frame sync, and telemetry identifier may be combined into a single entity such that the functionality of associated with these individual elements of the messages may be performed simultaneously or in series based on a single-but-repeated pattern of information.

The preamble (whether in the form of a standard or attention pattern) is used so that transceiver 150 (FIG. 3) can establish bit synchronization (i.e. bit-boundary recognition) of the incoming data. Additionally, the attention preamble can be used to obtain and retain the “attention” of implantable device 102 for a defined period of time. As long as the attention preamble is being received, transceiver 150 remains activated and continues tracking the signal in anticipation of an incoming message. In some examples, the preamble can adhere to minimum size requirements in order to reduce power consumption within implantable device 102. In other examples, the preamble used by implantable device 102 may be larger relative to the minimum number of bit transitions needed to establish bit synchronization.

The frame sync may actually be considered as a “byte sync” (e.g., frames are bytes) and is a single byte of a selected pattern such that the receiver can obtain byte boundaries for the transmitted data. For instance, the selected pattern could be “10110000.” Other patterns, pattern lengths, and frame sizes may be acceptable for use as a frame sync, so long as the patterns (in combination with preamble or attention preamble patterns and any tolerances in their reception) generally do not lead to errors in defining byte transitions. Even during the receipt of an attention preamble (or other preamble) the receiver continually compares shifting bits that are received to the anticipated frame-sync pattern. The frame-sync byte may be detected by feeding the incoming bit stream into a shift register and passing the bit values in the register to the inputs of an eight-input “AND” gate whose input-assertion levels correspond to the anticipated frame-sync pattern. This comparison process continues so long as the receiver continues to listen for an incoming message or until a valid frame-sync pattern has been received. Once frame sync is received, a valid frame-sync signal is asserted.

The telemetry identifier (or “telemetry ID”) is a value used to ensure that only the intended transceiver 150 receives a message. A unique ID is provided for each implantable device 102 during manufacturing. The telemetry IDs that transceiver 150 will consider “valid” are the ID of transceiver 150 or a universal ID communicable to a class of implantable devices 102. All other incoming bit patterns will be rejected such that transceiver 150 either turns off or restarts searching for a valid frame-sync pattern attention preamble. In other examples, if there is little risk of miscommunication, the telemetry ID may be dropped from the message protocol. In examples, the frame sync and telemetry ID may be combined into a single entity that is identified as a whole.

The data is provided in an integer number of bytes following the telemetry ID. In some examples, the first portion of the data can indicate the message type. For instance, the initial portion can be an operation code (or “op-code”), while the later portions can be either ignored or interpreted as a sequence number dependent on whether the first seven bits call for a sequence number. Each op-code can be followed by data in a defined number of bytes. The specific op-code itself may dictate the number of bytes that follow, or alternatively, the specific op-code may dictate that the number of bytes to follow may be extracted from the first byte or several bytes of information that follow it. In other examples, op-codes may have a different length, or the message length or message end may be dictated in other ways. Accordingly, based on the op-code and potentially one or more bytes following it, the receiver knows exactly how many more bytes of data to “listen” for. After receiving the bytes, transceiver 150 can turn off to conserve power.

Many different types of messages and responses thereto can be written into the programs that control implantable device 102. These messages may be used for a number of different purposes. For example, they may be: (1) system-level messages for testing devices, resetting devices, or establishing relationships between implantable device 102 and external devices 106, 108, 110; (2) “alarm” messages used to convey alarm conditions or to clear alarm conditions; (3) miscellaneous messages that set various parameters or perform various “read” operations; (4) “delivery” messages that set delivery amounts, read delivery status, or set parameters such as concentration and pump stroke volume necessary to appropriately control delivery of the drug; (5) data-log messages that set data-log boundaries, read boundaries, or clear data logs, boundaries, or read information from various data logs or supply information to those data logs; (6) “refill” messages related to an amount of material to be added to the reservoir periodically; (7) “compound messages” that perform more than one function, or (8) “error” messages that request error condition status or supply error condition status. In other examples, messages may be classified in different ways. In some examples, the messages may be downloaded from external devices 106, 108, 110 to implantable device 102. The downloading of software may include the downloading of executable software as well as the downloading of data structures that may be used by the executable software.

According to a telemetry timer (e.g., clock 154 of FIG. 3) of medical device 102, inbound transmissions begin at an “inbound-transmission start time” and continue for an “inbound transmission period” that defines the period that the preamble portion of the message is sent, which may be conceptualized either in terms of time or the number of data bits. Additionally, an “inbound-listening interval” is the interval between successive inbound-listening start times. Accordingly, transceiver 150 of implantable device 102 begins listening for messages at an “inbound-listening start time” and continues to listen for an “inbound-listening period.”

When communicating a message, it is considered safe practice to cease operation of implantable device 102, e.g., to avoid affecting a programming update and to ensure that the programming update is automatically applied. Specifically, ceasing operations serves to reduce the likelihood of a race condition, or other system-level hazard or error of computing device 120 as a result of one or more improperly sequenced functions. However, stopping implantable device 102 while a programming update is in-progress potentially creates an undesirable condition in which, when the telemetry update is interrupted without completion, implantable device 102 may unintentionally be left in a “paused” or otherwise inactive state. Such an interruption in the message transmission can be caused, for example, by external device 106, 108, 110 being physically moved out of position relative to implantable device 102 (e.g., out of an effective telemetry range), a “noisy” environment that makes telemetry communication difficult, interruption of the clinician by a colleague, the clinician changing their mind about the intended programming settings, etc.

To address this risk, techniques of this disclosure include a “timeout” module (e.g., configured to initiate a countdown timer) associated with a “cease operation” (e.g., “stop pump”) command, the timeout module being configured to notify the user (e.g., by sounding an alarm, etc.) should implantable device 102 not resume operations after a determined length of time needed for receipt and processing of the inbound message and subsequent restarting of therapy delivery (e.g., restarting of pump 118). Accordingly, the timeout module can serve as a mechanism to alert users about a state of device 102 shortly after or even during a programming update to reduce the likelihood of a drug-underdose scenario.

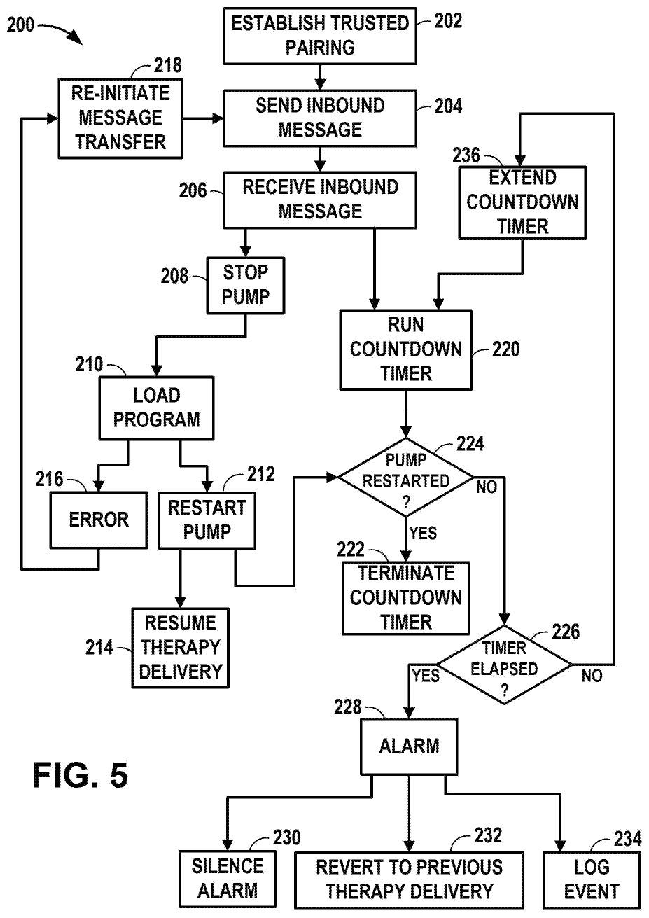

For instance, FIG. 5 is a flowchart 200 depicting a technique for executing a “timeout” module. A trusted pairing is established between implantable device 102 (FIG. 1) and one or more external device(s) 106, 108, 110 (202). An inbound message, e.g., in the form of a multi-part format or protocol, is sent by one of the external devices 106, 108, 110 (204) for subsequent receipt by implantable device 102 (206). The inbound message can include a “cease operation” command, e.g., configured stop delivery of the therapy by implantable device 102, regardless of current infusion prescription, boluses, etc. (208). Alternatively, implantable device 102 can revert to an established therapy-delivery rate (e.g., fixed-rate bolus, daily average rate, etc.).

The program or other data contained in the inbound message is loaded onto medical device 102 (210). If an error occurs during the loading process (216), the message-transfer process can be reinitiated (218), which prompts external device(s) 106, 108, 110 to resend the inbound message (204). Alternatively, if the programming is successfully loaded, operation of pump 118 of device 102 can be restarted (212), and therapy delivery resumes (214). Although the techniques of flowchart 200 contemplate ceasing operation of implantable device 102 prior to a programming update, the techniques can also be used to temporarily disable or cease therapy during diagnostic testing (e.g. magnetic resonance imaging, etc.), where exposure while operating implantable device 102 is not recommended.

In addition to the “cease operation” command (208), the inbound message can additionally include a “timeout” module configured to trigger a “Pump Stopped Too Long During Update” alarm, or other suitable notification, alert, or alarm should device 102 not be restated before a predetermined time duration expires. For example, the timeout module can begin a countdown timer for a specified period of time (e.g., 5 min., 10 min., 15 min., 20 min., 25 min., 30 min., etc.) (220).

In some examples, the timeout module can inquire whether the device operations have been restarted (224). If it is determined that device operations have been restarted (“YES” branch from 224), the countdown timer can be terminated (222). Conversely, if it is determined that restart has not occurred (“NO” branch from 224), the timeout module can determine whether the specified period of time has elapsed (226). If the specified period of time has not fully elapsed (“NO” branch from 226), the countdown timer can continue (236). Alternatively, at the conclusion of the specified period of time (“YES” branch from 226), a notification, alert, or alarm can be triggered (228). In other examples, this alert or alarm can be triggered upon reaching a predetermined condition being met or threshold being crossed, such as an empty reservoir, a motor stall, a low battery, transition to ERI state, etc. For instance, the alarm can be configured to provide an auditory signal, a tactile signal (e.g., vibration), or a visual signal (e.g., LEDs visible through the skin of patient 111) via implantable device 102. Additionally or alternatively to action by implantable device 102, the alarm can include various notifications, alerts, or alarms, including messages, auditory signals or tactile alarms, or the like, to one or more of external devices 106, 108, 110. Accordingly, should the programming update be interrupted and implantable device 120 fails to restart, the user will be alerted after expiration of the countdown timer. In some examples, implantable device 102 reverts to an established therapy-delivery rate (e.g., fixed-rate bolus, daily average rate, etc.) at the conclusion of the countdown timer (232). Thereafter, the user can determine the reason for the notification, alert, or alarm via external device(s) 106, 108, 110, and, if desired, either manually restart implantable device 102 or reinitiate transfer of the inbound message (218) to complete the programming update. Where transfer of the inbound message is reinitiated (218), the countdown timer can reset.

Alternatively to restarting implantable device 102 or reinitiating transfer of the inbound message (218), medical system 100 (or a user thereof) can terminate the countdown timer (222) upon determining that the countdown timer is not needed or is negatively affecting processing of the inbound message. Thus, implantable device 102 disarms the countdown timer (222) whenever operation of implantable device 102 is resumed or restarted (“YES” branch from 224).

In some examples, the notification, alert, or alarm can be silenced (230), e.g., after a defined period of time, or by user command, without restarting operation of implantable device 102. For instance, a patient may wish to silence the alarm until the “stopped pump” hazard can be addressed by a clinician. In other examples, the countdown timer can be extended (236), e.g., autonomously, or on-command, to enable additional time to complete receipt and processing of the inbound message. In some embodiments, operation of implantable device 102 can revert to a previous (e.g., most-recent) therapy-delivery schedule (232), e.g., to decrease the likelihood of a drug-underdose event. Where a notification, alert or alarm is triggered (228), the countdown time and any time thereafter can be automatically logged (234) to identify a gap in delivered therapy.

It should be understood that individual steps of the previous examples may be performed in any suitable order and/or simultaneously, as long as the overall technique remains operable. Similarly, various aspects disclosed herein may be combined in different combinations than those explicitly presented in the description and accompanying drawings. Additionally, certain aspects of this disclosure described as being performed by a single module or unit (e.g., for clarity) may also be performed by a combination of units or modules associated with a medical device 102 or computing device 106.

The techniques described herein may be implemented in hardware, software, firmware, or any suitable combination thereof. If implemented in software, the functions may be stored as instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital-signal processors (DSPs), general-purpose microprocessors, application-specific integrated circuits (ASICs), field-programmable logic arrays (FPGAs), or other equivalent integrated or discrete-logic circuitry. Accordingly, the term “processor” as used herein may refer to any of the foregoing structures or any other physical structure suitable for implementation of the described techniques, such as circuits or logic elements.

What is claimed is:

1. A system comprising:
an implantable medical device configured to deliver a therapy to a patient according to a therapy-delivery regimen; and
an external device configured to wirelessly transmit, to the implantable medical device, a programming update, a timeout module, and a command instructing the implantable medical device to cease a delivery of the therapy while installing the programming update, wherein, upon a failure of the implantable medical device to restart the delivery of the therapy after a duration of time indicated by the timeout module, the implantable medical device is configured to output an alert indicating that the implantable medical device has failed to restart.

2. The system of claim 1, wherein the implantable medical device comprises an infusion pump.

3. The system of claim 1, wherein the programming update comprises a software update for the implantable medical device or a modification for the therapy-delivery regimen.

4. The system of claim 1, wherein the implantable medical device is configured to generate and output an auditory signal, a tactile signal, or a visual signal comprising the alert.

5. The system of claim 1, wherein the implantable medical device is configured to wirelessly transmit the alert to the external device.

6. The system of claim 1, wherein the period of time comprises about 5 minutes, about 10 minutes, about 15 minutes, about 20 minutes, about 25 minutes, or about 30 minutes.

7. An implantable medical device comprising processing circuitry configured to:
deliver a therapy to a patient according to a therapy-delivery regimen;
wirelessly receive a programming update comprising a command instructing the implantable medical device to cease delivery of a therapy during installation of the programming update;
install the programming update and initiate a countdown timer for a predetermined duration of time; and
in response to failing to automatically restart delivery of the therapy upon expiration of the predetermined duration of time, generate and output an alert to notify a user that the implantable medical device has failed to restart.

8. The implantable medical device of claim 7, wherein the processing circuitry is configured to terminate the countdown timer upon restarting delivery of the therapy according to the therapy-delivery regimen.

9. The implantable medical device of claim 7, wherein the implantable medical device comprises an infusion pump.

10. The implantable medical device of claim 7, wherein the programming update is configured to update software of the implantable medical device or modify the therapy-delivery regimen.

11. The implantable medical device of claim 7, wherein the implantable medical device is configured to output an auditory signal, a tactile signal, or a visual signal comprising the alert.

12. The implantable medical device of claim 7, wherein the predetermined duration of time comprises about 5 minutes, about 10 minutes, about 15 minutes, about 20 minutes, about 25 minutes, or about 30 minutes.

13. A method comprising:
receiving, by an implantable medical device, a programming update comprising a command instructing the implantable medical device to cease a delivery of a therapy during installation of the programming update;
commencing, by the implantable medical device, an installation of the programming update;
upon commencing the installation of the programming update, initiating a countdown timer for a predetermined duration of time;
determining a failure to restart the delivery of the therapy upon expiration of the predetermined duration of time; and
outputting, by the implantable medical device, an alert to notify a user that the implantable medical device has failed to restart the delivery of the therapy.

14. The method of claim 13, further comprising, upon restarting the delivery of the therapy, terminating, by the implantable medical device, the countdown timer.

15. The method of claim 13, wherein the alert comprises an auditory signal, a tactile signal, or a visual signal.

16. The method of claim 13, wherein the implantable medical device comprises an infusion pump.
